(19) United States
(12) Patent Application Publication
(10) Pub. No.: US 2024/0165368 A1
(43) Pub. Date: May 23, 2024

(54) MODULAR SWITCHING-VALVE ASSEMBLY FOR A RESPIRATORY DEVICE

(71) Applicant: Hamilton Medical AG, Bonaduz (CH)

(72) Inventor: Daniel Hepting, Maladers (CH)

(21) Appl. No.: 18/281,818

(22) PCT Filed: Mar. 15, 2022
(86) PCT No.: PCT/EP2022/056638

§ 371 Date: Sep. 13, 2023

(30) Foreign Application Priority Data
Mar. 19, 2021 (DE) 10 2021 106 784.7

(51) Int. Cl. A61M 16/20; F16K 27/00; F16K 27/04

(52) U.S. Cl. CPC A61M 16/201; F16K 27/003; F16K 27/041; A61M 2202/0208

(57) ABSTRACT

A switching-valve assembly having a housing, first and second source ports, an outlet port, a first gas-supply line connecting the first source port to the outlet port, a second gas-supply line connecting the second source port to the outlet port, a valve arrangement in the housing adjustable between a first operating state, in which it opens the first gas-supply line to allow respiratory gas to flow through and blocks the second gas-supply line, and a second operating state, in which it blocks the first gas-supply line and opens the second gas-supply line to allow respiratory gas to flow through, the switching-valve assembly in the form of a modular switching-valve assembly being stackable, by the housing having a first outer-surface portion for stacking and a second outer-surface portion for stacking, which is designed to as to be complementary to the first outer-surface portion for stacking, wherein a source port is designed as a sequence port such that, when the first and the second outer-surface portions for stacking are in stacked engagement with one another, proceeding from the outlet and the sequence port, gas-supply line portions extending into the housing are each connected to a continuous gas-supply line region.

This Application claims priority in PCT application PCT/EP2022/056638 filed on Mar. 15, 2022, which claims priority in German Patent Application DE 10 2021 106 784.7 filed on Mar. 19, 2021, which are incorporated by reference herein.

The present invention concerns a switching-valve assembly for switching between two respiratory gas sources of a respiratory device, where the switching-valve assembly comprises:
A housing with a first source-linking aperture for connecting a first respiratory gas source, with a second source-linking aperture arranged spatially at a distance from the first one for connecting a second respiratory gas source and with an output-linking aperture arranged spatially at a distance from the first and from the second source-linking aperture for the output of respiratory gas from at least one of the source-linking apertures,
A first gas supply line which connects the first source-linking aperture with the output-linking aperture,
A second gas supply line which connects the second source-linking aperture with the output-linking aperture,
A valve arrangement accommodated in the housing which is displaceable between a first operational state in which it releases the first gas supply line for flow-through of respiratory gas and blocks the second gas supply line and a second operational state in which it blocks it the first gas supply line and releases the second gas supply line for flow-through of respiratory gas.

### BACKGROUND OF THE INVENTION

A switching-valve assembly with the features mentioned at the beginning is known from DE 29 18 791 A1. This known switching-valve assembly serves to supply a consumer connected to the output-linking aperture and not specified in further detail without interruption with gas from cylinder arrays which are connected to the first and to the second source-linking aperture.

Especially during the supply of artificially ventilated patients with respiratory gas, uninterrupted supply can contribute considerably to the success of treatment or even be life-saving.

Uninterrupted respiratory gas supply is first and foremost not self-evident if the respiratory gas cannot be extracted from a virtually infinitely large source, such as for example the ambient atmosphere, but rather the respiratory gas originates from a limited reservoir. This is the case in respiratory devices referred to as “hi-flow”, which independently from the respiratory rhythm and from the respiratory volume of the individual breaths of the patient supply respiratory gas continuously to the latter's mouth and/or nose. The respiratory gas, which often is supplied to a nasal cannula or mouth-nose mask carried by the patient, is then preferably oxygen or oxygen-enriched air, which exhibits a higher oxygen content than does the ambient atmosphere.

The switching-valve assembly known from DE 29 18 791 A1 teaches quite generally to utilize the gas pressures of the gas sources connected to the source-linking apertures in order to switch the valve arrangement automatically back and forth between its first and its second operational state. Thus, when the gas source at one source-linking aperture dwindles content-wise, whereas the gas source of the respective other source-linking aperture is still fully available, the depletion of the one gas source leads to a decreasing gas pressure in the gas supply line connected with this gas source such that the gas pressure of the other gas source which is then becoming greater relative to the gas pressure of the first gas source effects a displacement of the valve arrangement. The dwindling gas source is thereby disconnected fluid-mechanically from the output-linking aperture and the full gas source supplies to the output-linking aperture. The depleted gas source can then be exchanged against a fresh one which is then activated through its relatively greater becoming gas pressure when the latest activated gas source likewise begins to dwindle.

DE 29 18 791 A1 teaches to connect to each source-linking aperture not only one gas cylinder as a reservoir, but rather complete cylinder arrays with a plurality of gas cylinders in order to configure the exchange cycles of the individual gas sources connected to the source-linking apertures to be as long as possible.

This leads to two different drawbacks: First, the use of cylinder arrays leads to a relatively poor utilization of the individual cylinder filling, since the cylinder arrays of a gas source have to be connected among themselves via manifolds with one another, so that each individual cylinder of the cylinder arrays is connected with the assigned source-linking aperture and can supply gas to it. The dead space on the side of the gas source is thereby increased. Gas from the cylinders of a cylinder array can flow between the cylinders of the cylinder array and does not necessarily reach the output-linking aperture.

Second, the switching-valve assembly equipped with cylinder arrays quickly becomes unwieldy and can only be further used as a stationary switching-valve assembly. A configuration of the switching-valve assembly for a particular number of cylinders connectable to it can only take place via an appropriate structural design of the switching-valve assembly through appropriate use of manifolds which connect the desired number of cylinders with a source-linking aperture. Modification of a configuration once chosen is then only achievable through undesirably costly assembly work.

### SUMMARY OF THE INVENTION

In view of the drawbacks described above, it is the task of the present invention to improve the switching-valve assembly mentioned at the beginning in such a way that with it an arbitrary number of separate gas sources, in particular respiratory gas sources, can be connected to a consumer, preferably to a respiratory device, especially preferably to an aforementioned “hi-flow” respiratory device, where the individual gas sources should be reliably emptiable and a consumer supplied with more than two gas sources should continue to be usable as a mobile switching-valve assembly.

The present invention solves this task in the switching-valve assembly mentioned at the beginning by having the switching-valve assembly configured stackably as a modular switching-valve assembly, in that the housing exhibits a first outside section with a first stack exterior surface section and a second outside section arranged at a distance and/or at an angular distance relative to the first outside section with a second stack exterior surface section configured complementarily to the first stack exterior surface section, where the output-linking aperture is configured at an outside section out of the first and the second outside section and where a source-linking aperture out of the first and the second source-linking aperture is configured as a follow-up linking aperture at the respective other outside section out of the first and the second outside section, namely in such a manner that when the first and the second stack exterior surface sections are conceived as stack exterior surface sections of similar separate modular switching-valve assemblies in stack engagement with one another, gas supply line sections each extending from the outlet- and the follow-up linking apertures into the housing are connected into a continuous gas supply line region.

The switching-valve assembly of the present application is thus stackable with itself, i.e. two switching-valve assemblies designed according to the above task solution and therefore similar can be brought in such a manner into stack engagement with one another and thus stacked with one another that the output-linking aperture of the one switching-valve assembly can supply into a source-linking aperture of the respective other switching-valve assembly. The source-linking aperture following the output-linking aperture in the supply direction is therefore referred to in the present application as a “follow-up linking aperture”.

The design required for this type of stackability is, in like manner as in stackable toy bricks (for instance with Lego® bricks), readily ascertainable in an individual switching-valve assembly. The latter has to exhibit the first and the second stack exterior surface section at different locations, i.e. offset to one another, such that the first stack exterior surface section of this switching-valve assembly can be connected with the second stack exterior surface section of a similar switching-valve assembly, forming with one another a gas supply line region continuous across both switching-valve assemblies, and/or such that the second stack exterior surface section of this switching-valve assembly can be connected with the first stack exterior surface section of a similar switching-valve assembly, forming with one another a gas supply line region continuous across both switching-valve assemblies. Consequently, the first and the second stack exterior surface section of one and the same switching-valve assembly of the present application are in principle configured to be brought with one another into the aforementioned stack engagement, through which the gas supply line section leading to the output-linking aperture is connected with the gas supply line section going out from a source-linking aperture and leading into the housing into a common gas supply line region.

Thus the switching-valve assembly can be conceived to be divided into two assembly parts in such a way that each assembly part exhibits another exterior surface section of the two exterior surface sections with the respective assigned stack exterior surface section. These two assembly parts are then stackable with one another through establishing a stack engagement of the two stack exterior surface sections of the respective assembly parts.

Preferably the housings of stacked similar switching-valve assemblies form at least section-wise a common flush exterior surface. Preferably at least 75%, for preference at least 80%, of the length of a joint line discernible from outside of a joint formed between two stacked switching-valve assemblies are arranged in a common flush exterior surface formed by the two switching-valve assemblies.

In principle, to ensure the desired stackability it suffices if one gas supply line section going out from the output-linking aperture and proceeding into the housing and one gas supply line section going out from the follow-up linking aperture and proceeding into the housing form with one another, upon an established stack engagement, a continuous gas supply line region. Preferably the output-linking aperture and the follow-up linking aperture are arranged at the housing in such a manner that upon an established stack engagement they align with one another, which facilitates the sealing of the connection of output- and follow-up linking aperture against gas passage between the gas supply line region and the external environment.

In order to ensure stackability, the follow-up linking aperture and the output-linking aperture of one and the same switching-valve assembly can be penetrated through by one and the same aperture axis, where preferably the aperture axis proceeds in parallel to a stack axis along which a plurality of similar modular switching-valve assemblies are stackable. Preferably the follow-up linking aperture and the output-linking aperture are arranged concentrically to the aperture axis. Likewise, for reasons of the simplest possible fabrication, preferably the gas supply line sections which each proceed, going out from the follow-up linking aperture and the output-linking aperture, into the housing are penetrated through by the aperture axis, especially preferably concentrically to the aperture axis.

In the region of a connecting aperture there can be provided a connector formation for secure connection with a gas source or with a consumer as the case may be. The connector formation can be a screw thread, a part of a bayonet coupling, or a male or female part of a quick coupling for gas lines which is known per se. Preferably, every connecting aperture is provided with a connector formation. So that a connector formation does not collide with structures outside the switching-valve assembly during handling of the switching-valve assembly and get damaged, at least one connector formation, for preference all connector formations, of a switching-valve assembly are preferably arranged recessed into the housing from an outfall and/or an edge of the connecting aperture assigned to the connector formation.

As a result of the stack exterior surface sections being situated at an outside section of the housing, it is ensured that the complementary stack exterior surface sections of separate but similar switching-valve assemblies can be brought into engagement with one another.

In principle it is conceivable to arrange one or several modular switching-valve assemblies in a stack frame provided for that purpose, in particular in order to hold two or more modular switching-valve assemblies in stack engagement with one another. Such a stack frame as an additional component is, however, dispensable without loss of ability to secure the switching-valve assemblies in stack engagement if the switching-valve assembly exhibits fasteners for connecting with a similar modular switching-valve assembly. For example, the switching-valve assembly can exhibit an engagement projection and an engagement recess, where the engagement projection, for instance a moveable engagement hook, is configured for engagement with the engagement recess of a similar modular switching-valve assembly.

A simple but effective option for securing similar modular switching-valve assemblies in a stack engagement with one another can be realized by having at each outside section out of the first and the second outside section at least one connecting formation provided as a fastener, which is configured for securing a physical connection of the modular switching-valve assembly with a similar separate modular switching-valve assembly. Then modular switching-valve assemblies connected with one another to a stacked switching-valve assembly cascade can be directly connected with one another and secured to one another in stack engagement.

The stackability of a modular switching-valve assembly with a similar modular switching-valve assembly is manifested structurally in configuring the aforementioned connecting formations in that a relative spatial arrangement between the follow-up linking arrangement and the connecting formation in the outside section exhibiting the follow-up linking arrangement, is the same relative spatial arrangement which exists between the output-linking aperture and the connecting formation in the outside section exhibiting the output-linking aperture. Since the outside sections which after establishing a stack engagement abut against each other or lie opposite each other usually have to be configured as mirror-imaged to one another with respect to a mirror-symmetry plane orthogonal to the stack axis, the relative spatial arrangements of connecting formation and follow-up linking arrangement in the one outside section on the one hand and of connecting formation and output-linking aperture in the respective outside section on the other, can be present as mirror-imaged in a switching-valve assembly. This, however, does not alter the fact that the relative spatial arrangements, taking the mirror-symmetry into account, are the same, i.e. the at least one connecting formation and the connecting aperture in each of the two outside sections accommodating them are arranged in identical directions and at identical distances from one another.

The at least one connecting formation of the first outside section can be configured complementarily to the at least one connecting formation of the second outside section, for example as a projection and a recess, where the two connecting formations are fixable to one another, for example through screw threads, through a bayonet coupling, or through a combination of a latching projection with a latching recess. A connecting formation within the terms of the present application is also a passage aperture penetrating through the housing from the first to the second outside section, through which a fastener, such as for instance a threaded rod, a screw, or a clamping device and the like, can be passed. The passage apertures of switching-valve assemblies stacked along a stack axis preferably align, such that they are penetrated through by a common fastener and the switching-valve assemblies are securable to each other in the stacked state through one common fastener per passage aperture.

In order to be able to ensure that to a modular switching-valve assembly which is part of a bundle of stacked modular switching-valve assemblies there is connectable a respiratory gas source, preferably it is provided that the respectively other source-linking aperture out of the first and the second source-linking aperture which is not the follow-up linking aperture is provided in a different outside section of the housing than the first and the second outside section. The other outside section is still exposed even if the outside section exhibiting the follow-up linking aperture and the outside section exhibiting the output-linking aperture are situated in a stack of similar modular switching-valve assemblies and are covered by outside sections of other similar modular switching-valve assemblies.

A simple and effective stacking of two or more similar modular switching-valve assemblies with at the same time the smallest possible required installation space volume for the thus created stack can be made possible by the first and the second outside section being configured on opposite housing sides facing away from one another.

The respective other source-linking aperture can be configured in a third outside section which connects the first and the second outside section with one another. For example, the housing can exhibit a space-saving parallelepiped-like outer shape. Here the thinking is in particular about a die-shaped or cuboid housing shape, such that several similar modular switching-valve assemblies can be brought into stack engagement with one another into a stack with an unchanged footprint area through mere stacking on top of one another along a stack axis. Although this is not essential, in a preferred cuboid housing the first and the second outside section each forms a different end face of the housing. The third outside section can then be configured at one of the lateral surface sections. The end faces are here preferably the largest lateral areas of the cuboid housing, such that sufficient room is available both for the arrangement of the respective connecting aperture and for the arrangement of the aforementioned preferred at least one connecting formation.

In a die-shaped or cuboid housing, preferably the stack axis and the aforementioned aperture axis are oriented orthogonally to two opposite parallel outside sections.

Of course, to the follow-up linking aperture there can be connected a conventional respiratory gas source, for instance a gas cylinder, if the modular switching-valve assembly is used as the sole switching-valve assembly for supplying a respiratory device with respiratory gas.

Through the complementary designs of the first and the second stack exterior surface sections it is ensured that the first and the second stack exterior surface sections fit together to establish the stack engagement. The stack engagement can be a positive fit engagement of the first and second stack exterior surface sections and/or it can be an abutting engagement of the first and second stack exterior surface sections.

When between the first and the second stack exterior surface section it should be possible to establish a positive fit engagement as a stack engagement, the first stack exterior surface section can exhibit a formation out of a projection and a recess and the second stack exterior surface section can exhibit the respective other formation out of a projection and a recess complementarily to the formation of the first stack exterior surface section.

In the case of an abutting engagement as the stack engagement, the first stack exterior surface section and the second stack exterior surface section can each be planar or complementary curved stack exterior surface sections. If, according to a preferred further development, the modular switching-valve assembly exhibits at least one connecting formation in each of the first and the second outside sections, two connecting formations in each of the first and the second outside sections suffice for the correct relative orientation of modular switching-valve assemblies to be stacked with one another.

Likewise, the first and the second outside sections can be planar outside sections, as is the case in the aforementioned preferred parallelepiped-shaped housing. The stack axis and the aforementioned aperture axis are then preferably oriented orthogonally to the first and to the second outside sections.

In order to produce the most gas-tight continuous gas supply line region possible, there is provided at the outfall and/or at the edge, as the case may be, of at least one connecting aperture out of follow-up and output-linking aperture a sealing arrangement, for instance a sealing ring, which on establishing a stack engagement with a further similar modular switching-valve assembly comes into abutment against a structure of the further switching-valve assembly. Preferably there is arranged at both the follow-up and the output-linking aperture one sealing arrangement each. Preferably the structure of the further switching-valve assembly against which the sealing arrangement comes into abutment is then the sealing arrangement at the further switching-valve assembly. In order to achieve the best possible sealing effect, the surfaces facing outwards of the sealing arrangement at the output-linking aperture on the one hand and the sealing arrangement at the follow-up linking aperture on the other, can be configured to be complementary to each other such that they fit into each other along the lines of a lock and key principle, and when abutting against one another, additionally or alternatively to a sealing effect can form a kind of labyrinth seal through deforming abutment against each other.

In principle, the stack exterior surface sections can be designed arbitrarily. Preferably, however, they exhibit the connecting aperture respectively assigned to them spatially, such that aforementioned sealing arrangement is preferably arranged in the respective stack exterior surface section. It is, however, also possible to use the stack exterior surface sections only to ensure correct relative arrangement between two similar modular switching-valve assemblies. The correct relative arrangement then ensures that with an established stack engagement, the connecting apertures arranged in the respective outside sections at a distance from the complementary stack exterior surface sections and their gas supply line sections proceeding from the respective connecting apertures into the housing are connected with one another into a continuous gas supply line region going beyond the connecting apertures.

A switch valve arrangement of the type mentioned at the beginning, in particular the modular stackable switch valve arrangement described above, can undesirably enclose a gas volume under displacement of a valve body of its valve arrangement between its two operational positions which are assigned to the different aforementioned operational states of the valve arrangement in the region of the gas supply line which is to be blocked to through-flow. The enclosed gas volume can then, like a gas spring, act against the actually desired adjustment of the valve body into the desired operational position and in the most disadvantageous case prevent reaching the operational position or only allow brief reaching of the operational position and then move the valve body again away from the actually desired operational position.

The switching-valve assembly known from DE 29 18 791 A1 exhibits two separate valve bodies in the shape of translationally displaceable valve pistons, of which the valve piston lying nearer to the output-linking aperture opens via a mechanical tappet, in the region in which it blocks a gas supply line, a valve through which the blocked gas supply line is flushed with pressure-reduced gas which is derived downstream of the output-linking aperture from a gas line connected to the output-linking aperture downstream of a pressure reducer. In the switching-valve assembly known from DE 29 18 791 A1 too, due to its construction the enclosure of a gas volume in the region of the gas supply line to be blocked is possible with the aforementioned disadvantageous effects.

Additionally, when the initially blocked gas supply line is again released for a through-flow of gas, there exists in the known switching-valve assembly for a brief but definitely relevant duration a direct flow path from the gas source which is under very high pressure into the gas line leading to the consumer downstream of the pressure reducer. Such a flow path, even if existing only briefly, can in the event of ventilating a patient give rise to lethal effects.

In view of the disadvantages described above, the valve arrangement of a switching-valve assembly of the type mentioned at the beginning, which preferably contributes to supplying patients with respiratory gas, in particular of a modular stackable switching-valve assembly described above, exhibits a first valve body and a second valve body different from the first one, where the first and the second valve body are each arranged in the flow path between the first and the second source-linking apertures on the one hand and the output-linking aperture on the other, where the first valve body is accommodated displaceably in a first valve body cavity and where the second valve body is accommodated displaceably in a second valve body cavity arranged at a distance from the first valve body cavity, where the first and the second valve body are each displaceable between a first operational position assigned to the first operational state and a second operational position assigned to the second operational state, where the switching-valve assembly exhibits a first sealing arrangement effective between the first valve body and the first valve body cavity which in the first valve body cavity defines a first cavity region and a second cavity region different from the first one, where the switching-valve assembly exhibits a second sealing arrangement effective between the second valve body and the second valve body cavity which in the second valve body cavity defines a third cavity region and a fourth cavity region different from the third one, where a cavity region out of the first and third cavity region as first supply cavity region is part of the first gas supply line but not part of the second gas supply line, and where a cavity region out of the second and fourth cavity region as second supply cavity region is part of the second gas supply line but not part of the first gas supply line,

Where the switching-valve assembly exhibits a first venting line which connects the first supply cavity region with a first sink-linking aperture different from the output-linking aperture,

Where the switching-valve assembly exhibits a second venting line which connects the second supply cavity region with a second sink-linking aperture different from the output-linking aperture,

Where that other cavity region out of the first and third cavity region which is not first supply cavity region is as first venting cavity region part of the first venting line but not of the first gas supply line, and where that other cavity region out of the second and fourth cavity region which is not second supply cavity region is as second venting cavity region part of the second venting line but not of the second gas supply line.

Through the configuration of the first and the second venting line, which each lead to a sink-linking aperture, gas from the valve body cavity can be conducted away to a gas sink. Enclosure of a gas quantity in a critical cavity region through the valve body accommodated in the valve body cavity can thereby be prevented. To this end, the critical cavity region need only be part of the first or the second venting line. Since two operational positions of valve bodies exist in each of which a different gas supply line is blocked, there can also exist two critical cavity regions in which the enclosure of a gas quantity through the valve body acts against reaching the blocking operational position and/or remaining in it. Advantageously for venting the two critical cavity regions, each of these two critical cavity regions can be part of a different venting line out of the first or the second venting line.

In the simplest and therefore preferred case, the gas sink is the ambient atmosphere, such that the sink-linking aperture can be merely an aperture of the respective venting line into the ambient atmosphere.

It should, however, not be ruled out that to at least one sink-linking aperture there is connected a filter and/or a gas capture container.

The gas sink of the first venting line can differ from the gas sink of the second venting line. For a preferred ventilation application, however, normally it is sufficient if both gas sinks are the same gas sink. The first and the second sink-linking aperture can then be realized together in a single sink-linking aperture. The first and the second venting line can then exhibit a common venting line section in at least one region proximal to the common sink-linking aperture. For reasons of simpler fabrication, however, the separate configuration of the first and the second sink-linking aperture and of the first and the second venting line can be preferable. This is the case in particular when the first and the second venting line are configured as drilled venting lines and lead to venting cavity regions which lie spatially at a distance away from one another with a distance coordinate which runs transversely to the respective drilling axes of the venting lines.

In principle, each of the two valve bodies can be designed in such a way that on one functional side it blocks or releases a gas supply line and on its other functional side it influences a gas flow in a venting line. However, such a construction is complicated and requires adherence to certain constraints at the valve bodies. A more unambiguous functional allocation and greater structural freedom can be advantageously utilized when the first cavity region is the first supply cavity region, the second cavity region is the second supply cavity region, the third cavity region is the first venting cavity region, and the fourth cavity region is the second venting cavity region. Then both supply cavity regions are realized by one of the two valve body cavities and both venting cavity regions are realized by the respective other of the two valve body cavities. Consequently, one of the two valve bodies is then assigned to the two supply cavity regions and the respective other valve body is assigned to the two venting cavity regions. Thus every valve body cavity and every valve body accommodated in it can be configured and realized for the valve task intended for it.

In order to realize the valve arrangement and its venting function, a wall, in particular cavity wall, surrounding the first venting cavity region can surround a first venting valve body region of a valve body and a wall, in particular cavity wall, surrounding the second venting cavity region can surround a second venting valve body region of a valve body, where the first venting valve body region is configured to open or close the first venting line to a gas flow depending on the operational position of the valve body exhibiting the first venting valve body region, and where the second venting valve body region is configured to open or close the second venting line to a gas flow depending on the operational position of the valve body exhibiting the second venting valve body region. As already set out above, preferably the first and the second venting valve body region are configured at the same valve body.

Likewise, to secure the supply function of the valve arrangement, a wall, in particular cavity wall, surrounding the first supply cavity region can surround a first supply valve body region and a wall, in particular cavity wall, surrounding the second supply cavity region can surround a second supply valve body region, where the first supply valve body region is configured to block or release the first gas supply line to a gas flow depending on the operational position of the valve body exhibiting the first supply valve body region, and where the second supply valve body region is configured to block or release the second gas supply line to a gas flow depending on the operational position of the valve body exhibiting the second supply valve body region. As already set out above, preferably the first and the second supply valve body region are configured at the same valve body. This is preferably a valve body at which no venting valve body region is configured.

In principle it is conceivable for both the first and the second valve body to block the second venting line in their first operational position and for both to block the first venting line in their second operational position. Since, however, only a residual gas quantity is to be let out of a valve body cavity by venting, double closing of the venting lines by two or by both valve bodies, as the case may be, at the same time is unnecessary. Preferably, therefore, it is provided that out of the first and the second valve body only the valve body exhibiting the first venting valve body region is configured to open or to close the first venting line to a gas flow, and/or that out of the first and the second valve body only the valve body exhibiting the second venting valve body region is configured to open or to close the second venting line to a gas flow.

In principle it is conceivable for both the first and the second valve body to block the first gas supply line in their first operational position and for both to block the second gas supply line in their second operational position. It is precisely through the advantageous venting, which makes possible the valve arrangement of the switching-valve assembly being discussed here, that double blocking of the gas supply lines by two valve bodies is unnecessary. Preferably, therefore, it is provided that out of the first and the second valve body only the valve body exhibiting the first supply valve body region is configured to block or release the first gas supply line to a gas flow, and/or that out of the first and the second valve body only the valve body exhibiting the second supply valve body region is configured to block or release the second gas supply line to a gas flow.

For an especially installation-space-saving design, there can in the first supply valve body region be configured a first gas line which is part of the first gas supply line and there can in the second supply valve body region be configured a second gas line which is part of the second gas supply line. Additionally or alternatively, there can in the first supply valve body region be configured a first gas line which is part of the first venting line and there can in the second supply valve body region be configured a second gas line which is part of the second venting line. In order to be able to vent precisely that critical cavity region in which an enclosed gas quantity can exert an especially adverse effect on the positioning of the valve body, preferably the first gas line configured in the first supply valve body region is part both of the first gas supply line and also part of the first venting line. For the same reason, preferably the second gas line configured in the second supply valve body region is part both of the second gas supply line and of the second venting line.

Preferably, both the first and the second valve body are displaceable between their first and their second operational position only through the gas pressures prevailing in the switching-valve assembly. The first valve body and the second valve body and also the housing accommodating the two valve bodies can thereby be free from mechanical pre-tensioning devices acting on the first and the second valve body. This decreases the number of components of the switching-valve assembly and thereby the number of potential error sources and the number of potential servicing requirements.

As a switching-valve assembly for a respiratory device, the switching-valve assembly being discussed here is preferably also lubricant-free, such that no lubricant can be transported to the respiratory tract of a patient.

At least one of the two valve bodies can be a valve body displaceable rotationally about a valve body swivel axis between its operational positions. Preferably at least one of the two valve bodies is, especially preferably both valve bodies are, valve bodies displaceable translationally between their operational positions, most preferably valve pistons. For displacement between their operational positions only through gas pressures in the gas supply lines, at least one valve body is, preferably both valve bodies are, preferably a double-acting valve piston. Preferably every valve body is a double-acting valve piston, one of whose effective piston areas is acted upon by gas from the first gas supply line and whose oppositely-acting other effective piston area by gas from the second gas supply line.

The present invention further concerns a switching-valve arrangement, comprising a first switching-valve assembly and a second switching-valve assembly, each designed in accordance with the above description, where the first stack exterior surface section of the one switching-valve assembly out of first and second switching-valve assembly with the second stack exterior surface section of the respective other switching-valve assembly out of first and second switching-valve assembly are in stack engagement with one another, forming a gas supply line region continuous across the outlet linking arrangement of the one switching-valve assembly and the follow-up linking arrangement of the other switching-valve assembly.

The switching-valve arrangement preferably comprises a prioritization valve assembly which is known per se with a housing, with a first source-linking aperture configured at the housing for connecting a first gas source, with a second source-linking aperture prioritized relative to the first one configured at the housing for connecting a second gas source, and with an output-linking aperture configured at the housing for connecting a consumer, in order to supply the latter with gas from the first or the second gas source. The prioritization valve assembly comprises a first gas feed line which connects the first source-linking aperture with the output-linking aperture, and a second gas feed line which connects the second source-linking aperture with the output-linking aperture of the prioritization valve assembly. The prioritization valve assembly further comprises a prioritization valve arrangement, whose double-acting valve body exhibits differently dimensioned effective areas acting in opposite directions in such a way that it blocks the first gas feed line and opens the second gas feed line if to the second source-linking aperture there is connected a gas source with a predetermined minimum gas pressure, namely regardless of whether to the first source-linking aperture there is connected a predetermined gas source or not and regardless of the filling level of the predetermined gas source. With such a prioritization valve arrangement, pass-through precedence to the output-linking aperture of the prioritization valve assembly can always be accorded to a clinical building-installed respiratory gas source as the second gas source before a respiratory gas cylinder as the first gas source.

The prioritization valve arrangement is preferably stackable with a switching-valve arrangement as described above in such a way that the first source-linking aperture of the prioritization valve arrangement and the first gas feed line which starting from it proceeds into the housing of the prioritization valve arrangement, connect with the output-linking aperture of the switching-valve arrangement in a stacked state with the switching-valve arrangement.

The housing of the prioritization valve arrangement is preferably designed at least in one plane orthogonally to the stack axis with the same dimensions as the housing of the switching-valve arrangement, such that the prioritization valve arrangement and the switching-valve arrangement can form together an essentially flush exterior surface. The prioritization valve arrangement can exhibit for durable connection with the switching-valve arrangement at least one connecting formation which fits the at least one connecting formation of the switching-valve arrangement. Therefore, what was said above regarding the connecting formation of the switching-valve arrangement applies mutatis mutandis to the connecting formation of the prioritization valve arrangement.

The prioritization valve arrangement can be provided in the switching-valve arrangement additionally or alternatively to the aforementioned second switching-valve assembly.

The present invention further concerns a respiratory device, preferably mobile respiratory device for supplying a patient with respiratory gas, comprising a respiratory gas entry, a respiratory gas exit, a respiratory gas line which connects the respiratory gas entry with the respiratory gas exit, a flow modification device for modifying the respiratory gas quantity flowing through the respiratory gas exit per unit of time and/or for modifying the respiratory gas pressure at the respiratory gas exit, and a control device for controlling the operation of the flow modification device, where upstream of the respiratory gas entry the output-linking aperture of at least one switching-valve assembly designed in accordance with the above description or of a switching-valve arrangement designed in accordance with the above description is connected with the respiratory gas entry of the respiratory device in a gas-conveying manner.

These and other objects, aspects, features and advantages of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which forms a part hereof and wherein:

FIG. 1A perspective view of the housing of a switching-valve assembly according to the invention, looking at the output-linking aperture,

FIG. 2A longitudinal section through the housing along the section plane II-II which is parallel to the two end faces in FIG. 3 and FIG. 4,

FIG. 3A longitudinal section through the housing along the section plane III-Ill of FIG. 2 which is orthogonal to the end faces and parallel to the longitudinal portions of the lateral surface,

FIG. 4A plan view of the longitudinal portion of the lateral surface facing away from the observer in FIG. 1, when observing the housing along the arrow IV of FIG. 2,

FIG. 5A cross-section along the section plane V-V of FIG. 2 which is orthogonal to the end faces and parallel to the transverse portions of the lateral surface,

FIG. 6A plan view of the transverse portion of the lateral surface facing away from the observer in FIG. 1, when observing the housing along the arrow VI of FIG. 2,

FIG. 7A plan view of the end face of the housing facing away from the observer in FIG. 1, with a source-linking aperture which is a follow-up linking aperture of the housing,

FIG. 8A longitudinal section through the switching-valve assembly according to the invention with the valve arrangement in the first operational state, where the section plane through the switching-valve assembly is the same section plane as that of FIG. 2,

FIG. 9A longitudinal section according to FIG. 8 through the switching-valve assembly according to the invention with the valve arrangement in an intermediate operational state between the first operational state of FIG. 8 and the second operational state of FIG. 10,

FIG. 10A longitudinal section according to FIG. 8 through the switching-valve assembly according to the invention with the valve arrangement in the second operational state,

FIG. 11A longitudinal section according to FIG. 8 through the switching-valve assembly according to the invention with the valve arrangement in an intermediate operational state between the second operational state of FIG. and the first operational state of FIG. 8,

FIG. 12A perspective view of a stack out of switching-valve arrangements of FIGS. 1 to 11 and a prioritization valve assembly in a respiration system for uninterrupted supply of a patient with respiratory gas from several respiratory gas sources supplying successively,

FIG. 13A perspective exploded drawing of the stack of FIG. 12, and

FIG. 14A longitudinal section through follow-up and output-linking apertures in the stack of FIGS. 12 and 13 and through the gas supply line sections going out of them, which form a common continuous gas supply line region.

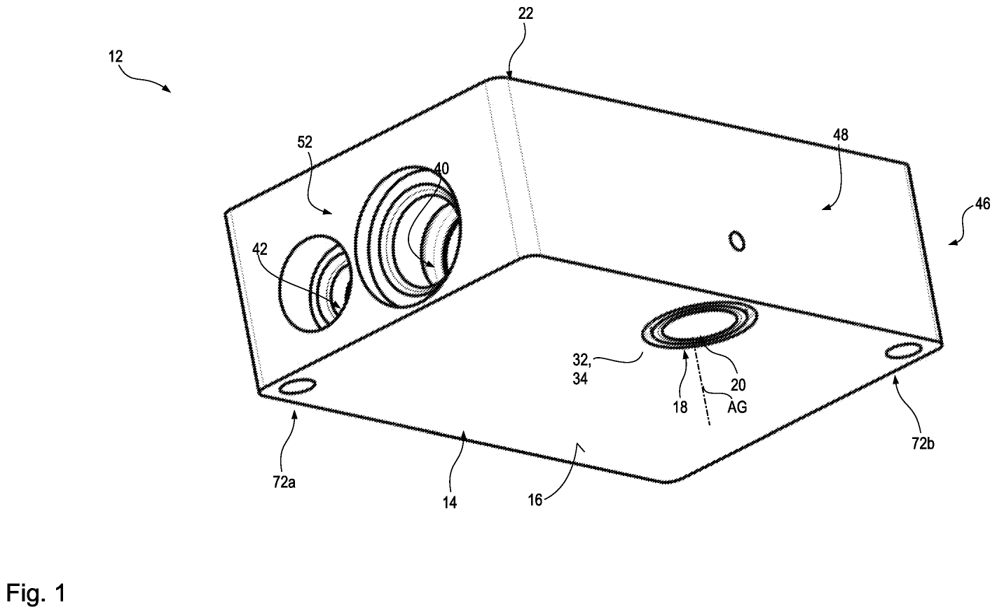

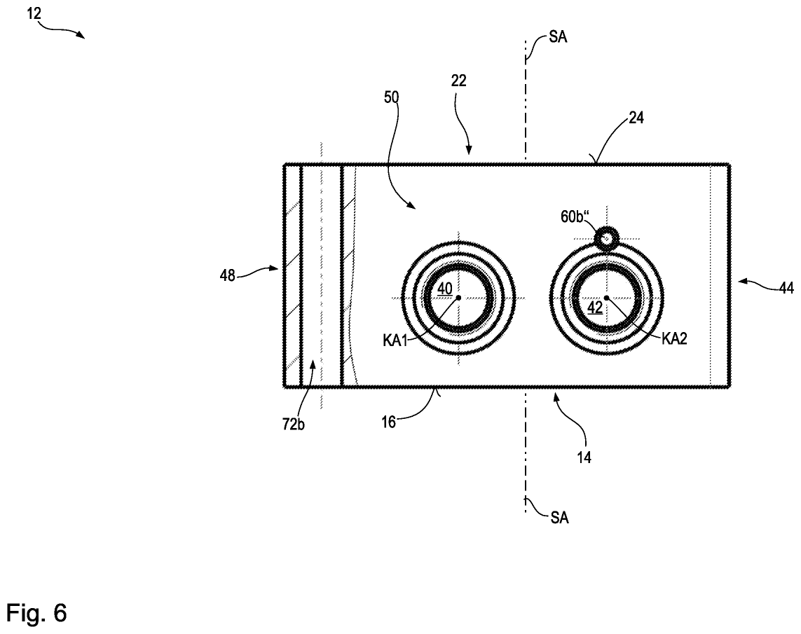

The complex structure of conduits inside the housing 12 of a switching-valve assembly according to the invention is first elucidated by reference to FIGS. 1 to 7 which should be considered together.

### DESCRIPTION OF PREFERRED EMBODIMENTS

Referring now to the drawings wherein the showings are for the purpose of illustrating preferred and alternative embodiments of the invention only and not for the purpose of limiting the same, FIG. 1 shows the housing 12 of a switching-valve assembly according to the invention in perspective from outside. The observer of FIG. 1 looks towards an end face as a second outside section 14. The outside section 14 exhibits a planar second stack exterior surface section 16 in which an output-linking aperture 18 is configured. The output-linking aperture 18 forms the end of an output gas supply line section 20, which in the depicted example extends along a virtual output gas supply path AG, which for reasons of simplified fabrication is a straight output gas supply path AG. The output gas supply path AG is oriented orthogonally to the stack exterior surface section 16.

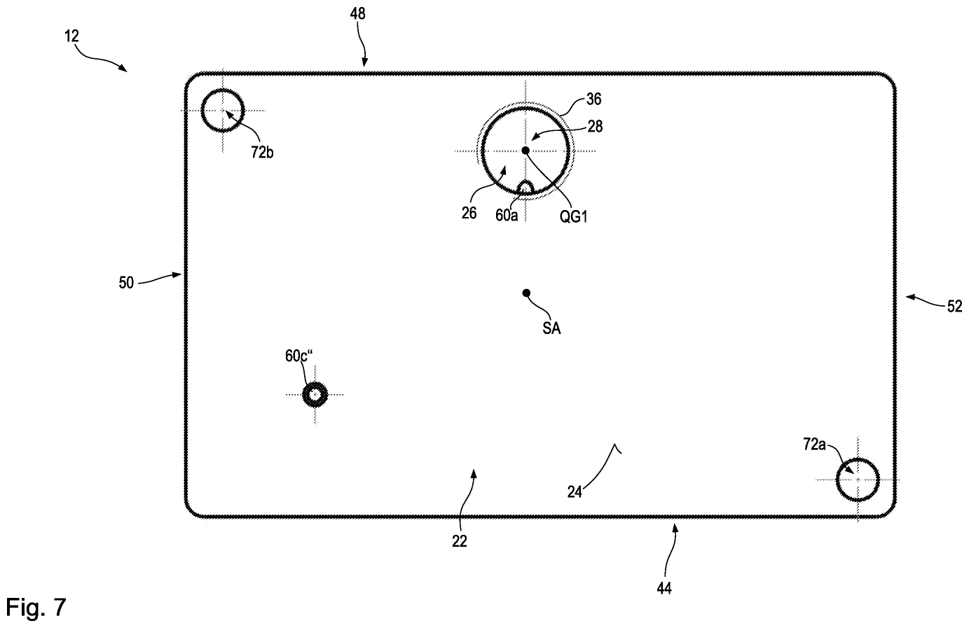

FIG. 7 shows the end face forming the first outside section 22, which exhibits a likewise planar first stack exterior surface section 24. The planar stack exterior surface sections 24 and 16 are parallel to one another and can be brought into abutment against one another in a planar manner when two housings 12 each with a stack exterior surface section 24 and 16 are being used.

In the first outside section 22 and also in the first stack exterior surface section 24 there is configured a first source-linking aperture 26, going out from which a first source gas supply line section 28 proceeds along a virtual first source gas supply path QG1 into the housing 12. The first source gas supply path QG1 is straight and oriented orthogonally to the first stack exterior surface section 24.

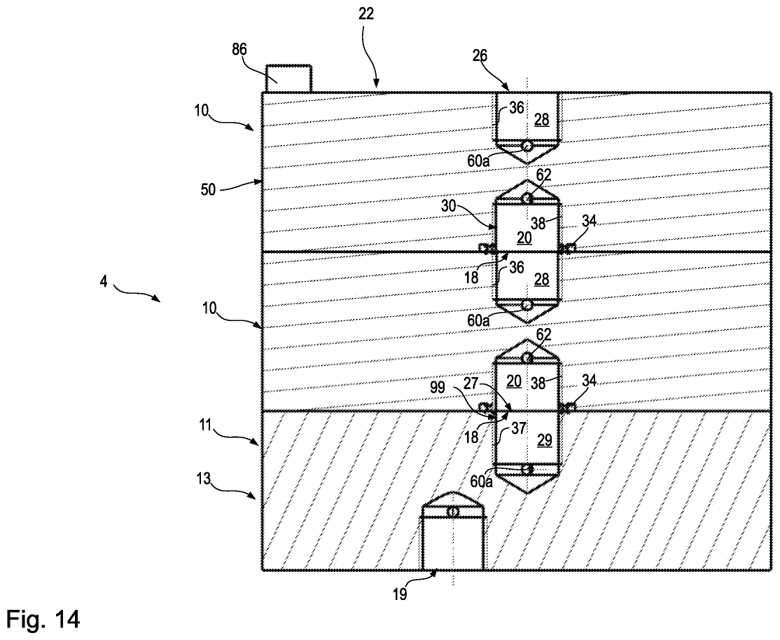

When two similar switching-valve assemblies or housings 12 as the case may be are stacked with one another in stack engagement along a stack axis SA in such a way that the first stack exterior surface section 24 and the second stack exterior surface section 16 abut against one another, the first source-linking aperture 26 and the output-linking aperture 18 can align with one another. The first source-linking aperture 26 is therefore a follow-up linking aperture 26 within the meaning of the foregoing introductory description. Therefore, the first source-linking aperture 26 and the output-linking aperture 18 are arranged along mutually coaxial gas supply paths: first source gas supply path Q1 and output gas supply path AG, which form a common aperture axis. The first source gas supply line section 28 and the output gas supply line section 20 too, align with one another in the described stack engagement and form through the aligned connecting apertures 26 and 18 a common gas supply line region 30 (see FIG. 14).

The output-linking aperture 18 is surrounded by a sealing arrangement 32 in the shape of an O-ring, which is arranged in a groove 34 configured at a radial distance from the output-linking aperture 18 concentrically to the output-linking aperture 18. The sealing arrangement 32 abuts against the first stack exterior surface section 24 in a sealing manner, in stack engagement with a first stack exterior surface section 22 of a housing 12.

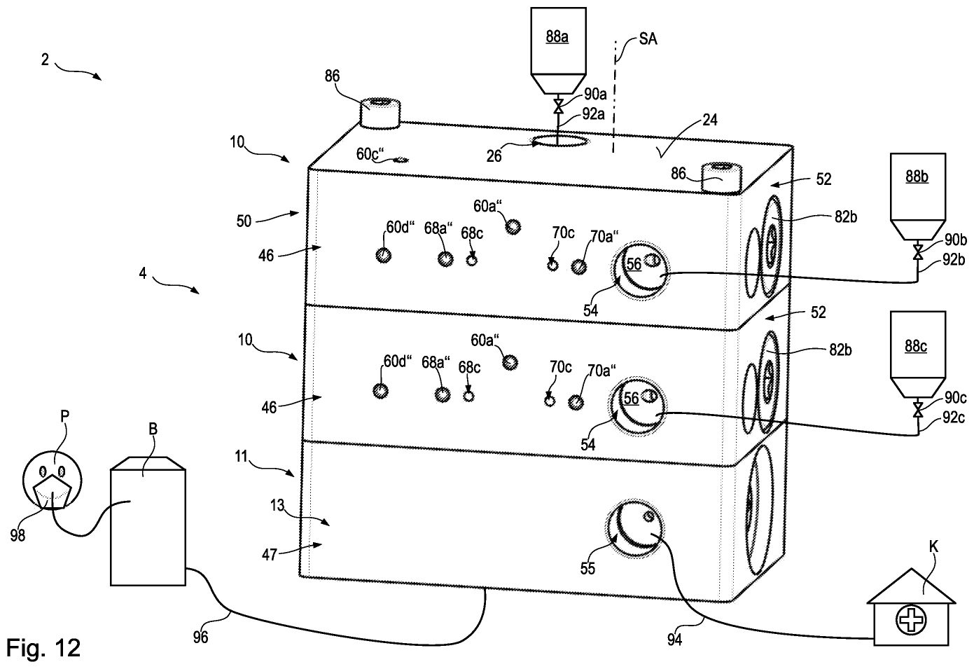

For securing a first gas source 88a (s. FIG. 12), for instance a conduit hose 92a (s. FIG. 12) leading from a gas cylinder 88a to the housing 12, there is configured at the first source-linking aperture 26 and in the first source gas supply line section 28 a first source connector formation 36 in the shape of an internal thread. Into this there can be screwed a connecting piece of a conduit hose 92a or the like.

Likewise, for securing a gas line, again for instance a conduit hose 96 (s. FIG. 12), leading to a consumer, there is configured at the output-linking aperture 18 and in the output gas supply line section 20 an output connector formation 38, likewise in the shape of an internal thread. Into this there can be screwed a connecting piece of a conduit hose 96 leading to a consumer or the like.

Instead of the depicted internal thread, bayonet coupling formations or pneumatic quick couplings can also be configured as connector formations.

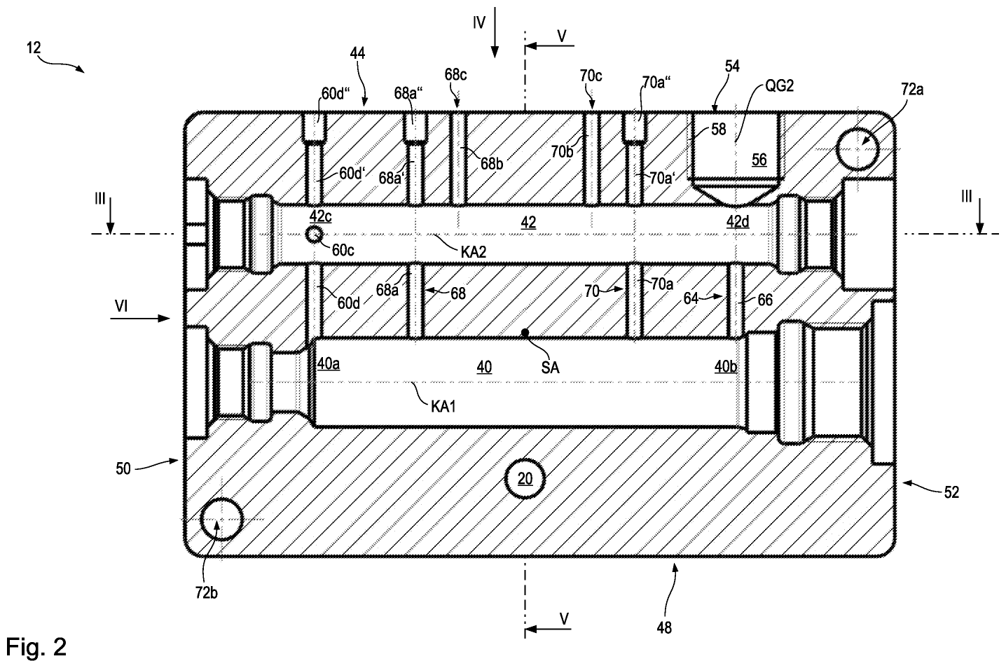

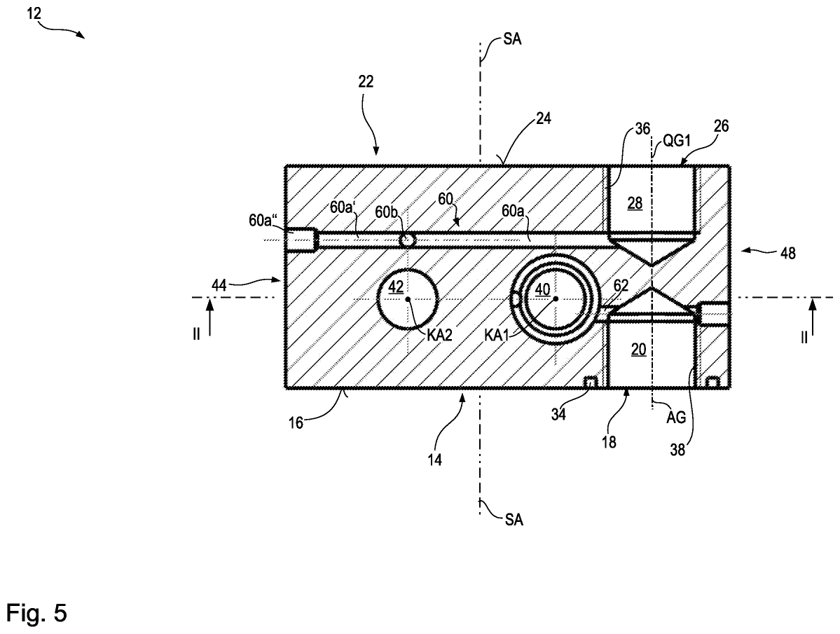

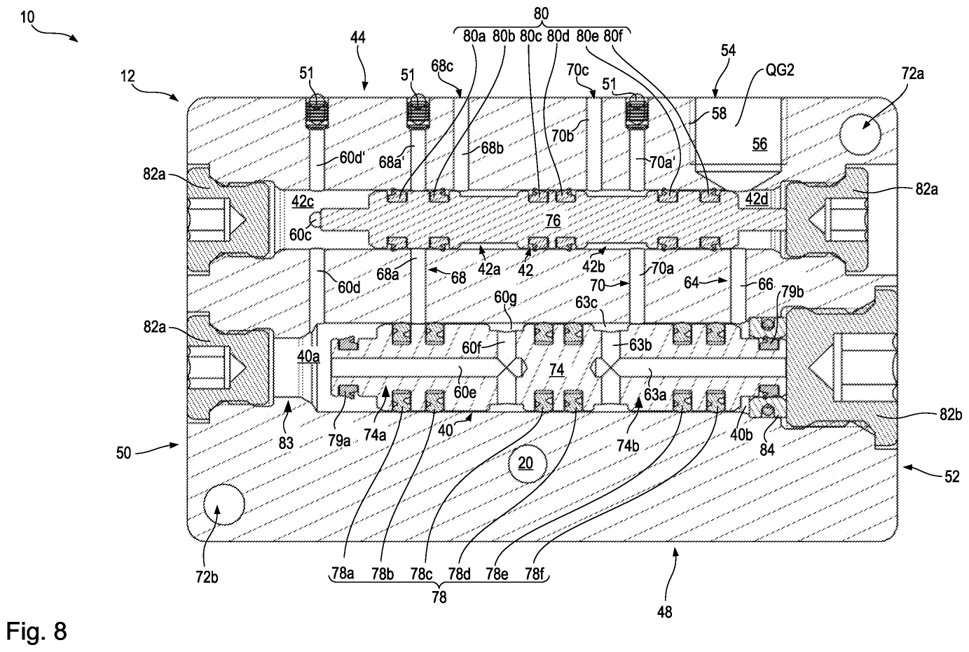

As can be discerned in FIGS. 2 and 5, the housing 12 exhibits a first valve body cavity located nearer to the output-linking aperture 18 and/or the output gas supply line section 20 respectively and a second valve body cavity 42 located further away from the output-linking aperture 18 and/or the output gas supply line section 20 respectively. The first valve body cavity 40 extends along a straight first cavity axis KA1. The second valve body cavity 42 extends along a straight second cavity axis KA2 parallel to the first cavity axis KA1. The parallelism of the cavity axes KA1 and KA2 is not essential, but practical. In the valve body cavities 40 and 42 there are accommodated displaceably at the ready-to-use switching-valve assembly 10 a first and a second valve body 74, 76 respectively (see FIGS. 8 to 11).

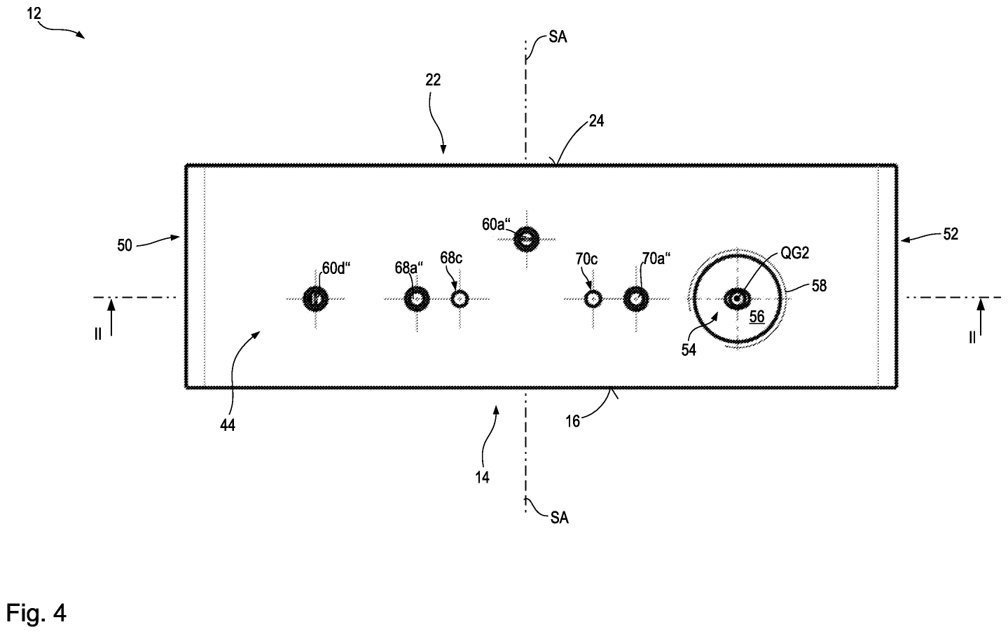

The observer of FIG. 4 looks towards the longitudinal portion 44, which in FIG. 1 faces away from the observer, of the lateral surface 46 which encircles between the end faces. The longitudinal portion 44 forms a third outside section which runs between the first outside section 22 and the second outside section 16, in the depicted example of the cuboid housing 12 orthogonally to those.

Parallel to the longitudinal portion 44 there runs the further longitudinal portion 48 discernible in FIG. 1. The two longitudinal portions 44 and 48 are connected with one another by two transverse portions 50 and 52 of the lateral surface 46 which likewise are parallel to one another and orthogonal to the end faces.

In the longitudinal portion 44, i.e. the third outside section 44, there is configured a second source-linking aperture 54, which in its shape equals the first source-linking aperture 26. This has the advantage that a gas source which is connectable to the first source-linking aperture 26 is also connectable to the second source-linking aperture 54 and vice versa. From the second source-linking aperture 54 there thus extends a second gas supply line section 56 along a virtual second source gas supply path QG2, which like the aforementioned gas supply paths QG1 and AG is configured as a straight source gas supply path QG2. The second source gas supply path QG2 is oriented in the depicted example orthogonally to the first source gas supply path QG1 and to the output gas supply path AG. For securing a connection between a gas source, which can also be a gas line, and the housing 12 there is configured in the second gas supply line section 56 a second source connector formation 58 in the shape of an internal thread. What was said regarding the first source connector formation 36 also applies to the second source connector formation 58.

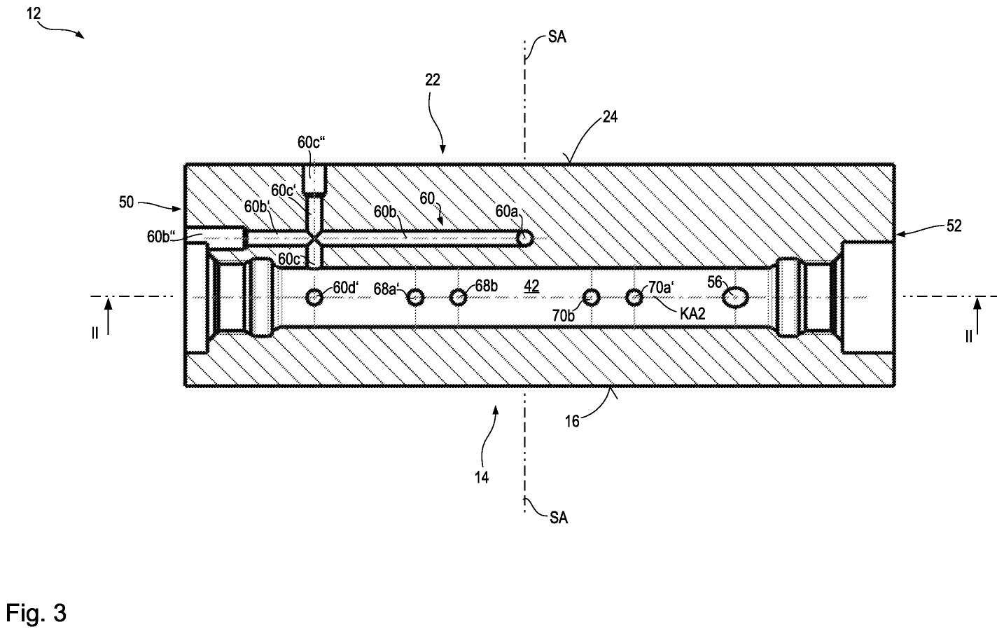

The first gas supply line 60 is elucidated hereunder: The first gas supply line 60, to which the first gas supply line section 28 already belongs, comprises in the depicted example a further line section 60a, which extends from the first gas supply line section 28 parallel to the first and the second outside section 24 and/or 16 as the case may be and in addition parallel to the transverse portions 50 and 52 of the lateral surface 46 past the valve body cavities 40 and 42 (see FIG. 3). The further line section 60a is manufactured, by way of example, as a drilled hole, which starting from the longitudinal portion 44 is led orthogonally to the latter up to the first gas supply line section 28.

To the line section 60a there is connected orthogonally to it and parallel to the second valve body cavity 42 and/or parallel to the second cavity axis KA2 respectively a line section 60b, which by way of example is implemented as a drilled hole starting from the transverse portion 50.

Finally, the first gas supply line 60 exhibits a third line section 60c orthogonal both to the line section 60a and to the line section 60b, which eventually opens into a first longitudinal end region 42c of the second valve body cavity 42 located nearer to the transverse portion 50. Starting from there, a further line section 60d of the first gas supply line 60 leads to a first longitudinal end region 40a of the first valve body cavity 40 located nearer to the transverse portion 50. From the first valve body cavity 40, namely approximately from its longitudinal center, a line section 62 (s. FIG. 5) of the first gas supply line 60 leads to the output gas supply line section 20. Thus gas from the first source-linking aperture 22 can flow along the first gas supply line 60 up to the output-linking aperture 18.

The second gas supply line 64 is described hereunder (see FIG. 2): The second source gas supply line section 56 extending from the second source-linking aperture 54 into the housing 12 is a first section of the second gas supply line 56. This second source gas supply line section 56 opens directly into a longitudinal end region 42d of the second valve body cavity 42 located nearer to the transverse portion 52. From this longitudinal end region 42d of the second valve body cavity 42, a further line section 66 leads to a longitudinal end region 40b of the first valve body cavity 40 located nearer to the transverse portion 52. From the first valve body cavity 40, the line section 62, which thus is part both of the first gas supply line 60 and of the second gas supply line 64, leads to the output gas supply line section 20, which likewise is part both of the first gas supply line 60 and of the second gas supply line 64.

The line section 62 lies in front of the section planes of FIGS. 2 and 8 to 11.

The line sections 60d and 66 thus proceed parallel to one another between opposite longitudinal end regions of the first valve body cavity 40 and the second valve body cavity 42.

In the housing 12 there are configured besides a first venting line 68 and a second venting line 70. The first venting line 68 is situated nearer to the transverse portion 50 and preferably proceeds in parallel to it and also parallel to the end faces. The second venting line 70 is situated nearer to the transverse portion 52 and preferably proceeds in parallel to it and also parallel to the end faces.

The first venting line 68 comprises a first line section 68a, which preferably proceeds in parallel to the line section 60d but nearer to the longitudinal center of the first valve body cavity 40 between the first valve body cavity 40 and the second valve body cavity 42 and connects the cavities with one another.

The first venting line 68 comprises moreover a second line section 68b which proceeds from the second valve body cavity 42 to a sink-linking aperture 68c and thereby opens towards the external environment of the housing 12. In the depicted example, the sink-linking aperture 68c is situated in the longitudinal portion 44 of the lateral surface 46. The outfall of the second line section 68b into the second valve body cavity 42 is arranged offset along the second cavity axis KA2 relative to the outfall of the first line section 68a into the second valve body cavity 42, namely in the depicted example in the direction towards the longitudinal center of the second valve body cavity 42.

The second venting line 70 comprises a first line section 70a, which preferably proceeds in parallel to the line section 66 but nearer to the longitudinal center of the first valve body cavity 40 between the first valve body cavity 40 and the second valve body cavity and connects the cavities with one another.

The second venting line 70 comprises moreover a second line section 70b which proceeds from the second valve body cavity 42 to a sink-linking aperture 70b and thereby opens towards the external environment of the housing 12. In the depicted example, the sink-linking aperture 70c is situated in the longitudinal portion 44 of the lateral surface 46. The outfall of the second line section 70b into the second valve body cavity 42 is arranged offset along the second cavity axis KA2 relative to the outfall of the first line section 70a into the second valve body cavity 42, namely in the depicted example in the direction towards the longitudinal center of the second valve body cavity 42.

The two second sections 68b and 70b of the first and the second venting lines 68 and 70 respectively, consequently exhibit a smaller distance from one another along the second cavity axis KA2 than the first two sections 68a and 70a.

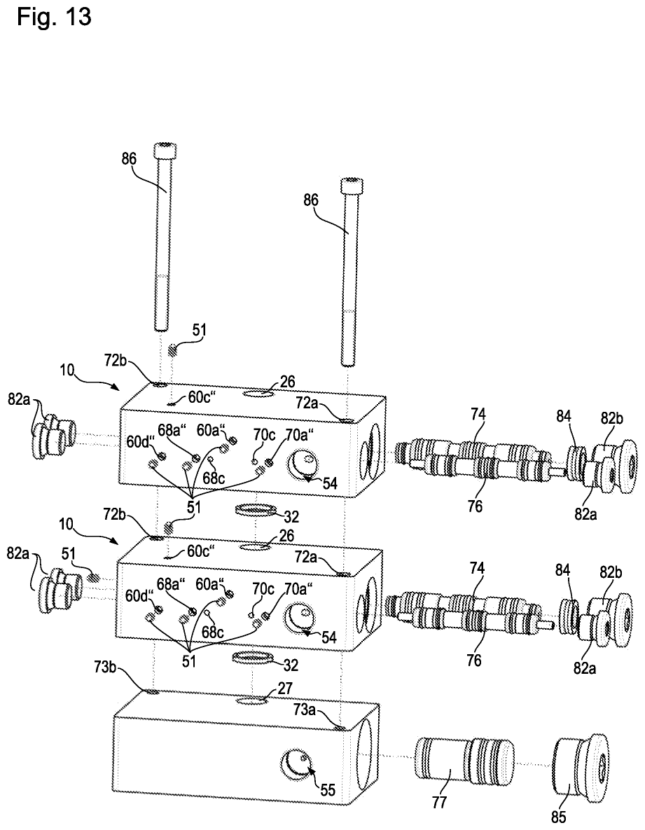

At each of two diagonally opposite corners of the in total four corners of the housing 12 there is configured a connecting formation 72a and 72b respectively in the shape, for example, of a through-bore or a through-duct as the case may be. Through each of the connecting formations 72a and 72b there can be passed for example a bolt 86 (s. FIGS. 12 and 13) or a threaded rod or some other clamping device, in order to connect two or more housings 12 with one another along a stack axis SA orthogonal to the end faces and secure them to one another. The connecting formation 72a is situated in the housing corner formed by the transverse portion 52 and the longitudinal portion 44. The connecting formation 72b is situated in the opposite housing corner formed by the transverse portion 50 and the longitudinal portion 48. The connecting formations can be configured in more than two corners, in a different pair of corners, or even elsewhere than in housing corners, to mention only a few possible variations.

Generally speaking, those sections lying in the extension of line sections, which themselves fulfill no conduit function but were formed only in order to produce the assigned coaxial line section, are labelled with the same reference mark as the assigned coaxial line section but with the addition of an apostrophe. These apostrophe-labelled fabrication sections are configured at the outside section into which they open, with an end section having an enlarged diameter in order to be able to arrange inside it a closure 51 of the apostrophe-labelled fabrication sections which seals the fabrication sections against the external environment. The end sections with enlarged diameters are labelled with the same reference mark as the assigned coaxial line section but with the addition of a double apostrophe.

FIG. 8 shows the complete switching-valve assembly 10, with a first valve body 74 accommodated in the first valve body cavity 40 and with a second valve body 76 accommodated in the second valve body cavity 42. The first and the second valve body 74 and 76 respectively are translationally displaceable, double-acting valve pistons, which are acted upon at their respective longitudinal ends by gas from the first gas supply line 60 and from the second gas supply line 64. In FIG. 8, the switching-valve assembly 10 is depicted in its first operational position in which the first gas supply line is released for flow-through of gas from the first source-linking aperture 26 to the output-linking aperture 18 and in which the second gas supply line 64 is blocked to flow-through of gas from the second source-linking aperture 54 to the output-linking aperture 18. Likewise, the first venting line 68 is blocked and the second venting line 70 is opened for flow-through.

The first valve body 74 and the second valve body 76 are in their first operational position.

The first valve body 74 exhibits a first sealing arrangement 78 which in the depicted example comprises from left to right six sealing rings 78a to 78f. The sealing rings 78a to 78f are arranged at the first valve body 74 for common movement with the first valve body 74 and seal against the inner wall of the first valve body cavity 40.

The second valve body 76 likewise exhibits a second sealing arrangement 80 which in the depicted example comprises from left to right six sealing rings 80a to 80f. The sealing rings 80a to 80f are arranged at the second valve body 76 for common movement with the second valve body 76 and seal against the inner wall of the second valve body cavity 42.

The first sealing arrangement 78, in particular the seals 78c and 78d, defines in the first valve body cavity 40 the first cavity region 40a and the second cavity region 40b which disconnected fluid-mechanically from the first one. The first cavity region 40a is, as a first supply cavity region 40a, part of the first gas supply line 60 but not of the second gas supply line 64. The second cavity region 40b is, as a second supply cavity region 40b, part of the second gas supply line 64 but not of the first gas supply line 60.

In the first valve body 74 there are configured, at its left end in FIG. 8, in the first supply valve body region 74a of the first valve body 74, a line section 60e proceeding along the longitudinal axis of the first valve body 74 and a line section 60f proceeding orthogonally thereto. The line section 60f opens into a ring line 60g encircling the longitudinal axis of the first valve body 74 in the circumferential direction. The line sections 60e, 60f, and 60g configured in or at the first valve body 74 are part both of the first gas supply line 60 and of the first venting line 68.

Likewise there are configured in the first valve body 74, which is configured in a mirror-imaged manner with respect to a median longitudinal plane, at its right end in FIG. 8, in a second supply valve body region 74b of the first valve body 74, a line section 63a proceeding along the longitudinal axis of the first valve body 74 and a line section 63b proceeding orthogonally thereto. The line section 63b opens into a ring line 63c encircling the longitudinal axis of the first valve body 74 in the circumferential direction. The line sections 63a, 63b, and 63c configured in or at the first valve body 74 are part both of the second gas supply line 64 and of the second venting line 70.

At the finished switching-valve assembly 10, the valve body cavities 40 and 42 are closed off at their respective longitudinal ends with locking nuts 82a and 82b. The three locking nuts 82a are identical. The locking nut 82b is larger than the locking nut 82a since through the aperture closed off by it, the first valve body 74 is introduced into the first valve body cavity 40. For the same reason, a constriction 83 which is configured at the left end in FIG. 8 of the first valve body cavity 40 integrally with the housing 12, which exhibits a smaller diameter than the rest of the central region of the first valve body cavity 40, is replicated at the right end in FIG. 8 of the first valve body cavity 40 by a separately configured and installed socket 84.

The sealing arrangement 78, in particular the seals 78a and 78b on the one hand and the seals 78e and 78f on the other, isolate in the first operational position of the first valve body 74 shown in FIG. 8 the line section 68a of the first venting line 68 from the rest of the first valve body cavity 40, thus preventing gas flow from the first valve body cavity 40 to line sections of the first venting line 68 located downstream in the venting direction.

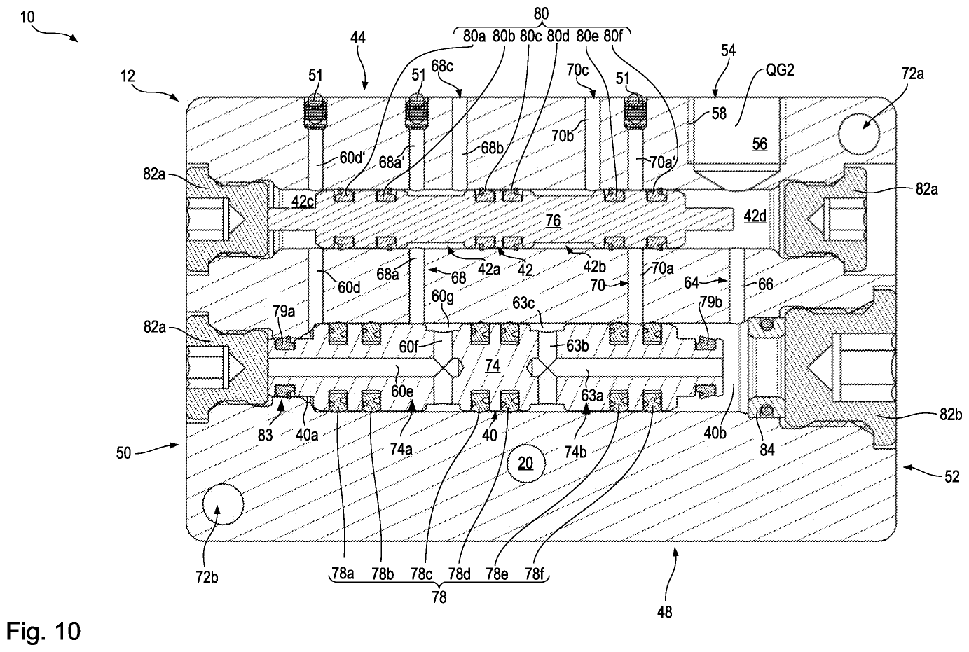

The sealing arrangement 78, in particular the seals 78e and 78f, do the same in the second operational position of the first valve body 74 (see FIG. 10) with the line section 70a and with the line sections of the second venting line 70 located downstream of the first valve body cavity 40 in the venting direction, respectively. In the first operational position of the first valve body 74 shown in FIG. 8, the seals 78e and 78f have no particular function.

The second sealing arrangement 80, in particular the seals 80c and 80d, isolates at the second valve body 76 in its first venting valve body region 76a and/or in the second valve body cavity 42 respectively a third cavity region 42a and a fourth cavity region 42b located in a second venting valve body region 76b of the second valve body 76 from one another. The third cavity region 42a is as first venting cavity region 42a part of the first venting line 68, but is not part of the second venting line 70 or of one of the gas supply lines 60 and 64. The fourth cavity region 42b is as second venting cavity region 42b part of the second venting line 70, but is not part of the first venting line 68 or of one of the gas supply lines 60 and 64. The cavity regions 42a and 42b are configured axially as a distance from one another and each proceed in an annular manner about the second valve body 76.

The second sealing arrangement 80, in particular the seal 80a on the one hand and the seal 80f on the other, isolate the cavity regions 42c and 42d on the longitudinal end side from the third and from the fourth cavity region 42a and 42b respectively. The cavity regions 42c and 42d on the longitudinal end side are part of the first gas supply line 60 and of the second gas supply line 64 respectively and are always, regardless of the position of the second valve body 76, capable of flow-through from the source-linking aperture, at which they end, towards the output-linking aperture 18. The second valve body 76 thus blocks in the depicted preferred embodiment example, in none of its operational positions, a gas supply line 60 or 64, but merely releases a venting line for flow-through and blocks the respective other venting line.

In the first operational position of the second valve body 76 shown in FIG. 8, the first sealing arrangement 80, in particular the seals 80a and 80b, isolates the line section 68a of the first venting line 68 from the rest of the valve body cavity 42, thus separating the line section 68a from the line section 68b, whereby the first venting line 68 is interrupted by the second valve body 76. In contrast, gas can escape from the second cavity region 40b via the line sections 63a, 63b and the ring line 63c into the line section 70a and from there via the fourth cavity region 42b into the line section 70b and eventually via the sink outlet aperture 70c from the housing 12 into the ambient atmosphere.

Thus in the depicted first operational position of the second valve body 76, no gas cushion can be enclosed in the second cavity region 40b which would be likely to force the first valve body 74 out of its first operational position shown in FIG. 8 in the direction towards it second operational position to the left.

In the first operational position of the second valve body 76, gas can get from the first source-linking aperture 26 via the line sections 60a, 60b, 60c, the longitudinal end side cavity region 42c, the line section 60d, the first cavity region 40a, and via the line sections 60e and 60f configured inside the first valve body 74 and via the ring line 60g into the line section 20 and thus into the gas supply line section 20 and to the output outlet aperture 18.

The end-side piston areas of the first valve body 74 are each configured in two parts, where a radially inner disk-shaped piston area section is arranged at an axial distance from a radially outer annular piston area section. Axially between the two piston area sections of one and the same piston area there is arranged in each case a seal 79a or 79b respectively. In the second operational position of the first valve body 74, The seal 79a abuts against the constriction 83 and together with the sealing arrangement 78, in particular the seal 78a, interrupts in the second operational position the first gas supply line 60. In the first operational position of the first valve body 74 depicted in FIG. 8, the seal 79b abuts against an inner circumferential area of the socket 84, thus blocking together with the sealing arrangement 78, in particular the seal 78f, the second gas supply line 64. In the first operational position of the first valve body 74 shown in FIG. 8, gas from the second source-linking aperture 54 can flow only up to and into the second cavity region 40b, but no further.

Through the division of the two piston areas and through the separation of the radially inner disk-shaped piston area section by the seals 79a and 79b respectively on the side of the respectively deactivated, blocked gas supply line, hysteresis of the first valve body 74 is effected which prevents ‘flutter’ of the first valve body 74 when the magnitudes of the pressures prevailing in the gas supply lines 60 and 64 are approximately equal pressures.

The two ring lines 60g and 63c are each so designed that in one of the two operational positions of the first valve body 74 they establish a flow connection with the line section situated in front of the drawing plane of FIGS. 2 and 8 to 11 and in the respective other of the two operational positions they establish a flow connection with a respective other out of the line sections 68a and 70a.

Similarly to the first valve body 74, the piston areas at the the longitudinal end regions of the second valve body 76 are also configured in two parts. Each of the piston areas exhibits a radially inner disk-shaped piston area section and a radially outer annular piston area section offset axially to it. Hereby too, hysteresis of the second valve body is achieved for the case that in the two end-side cavity regions 42c and 42d there prevail gas pressures similar or equal in magnitude. The radially inner disk-shaped piston area section which in an operational position of the second valve body 76 abuts against a locking nut 82a, is not reached by gas in the respective cavity region 42c or 42d, such that the gas pressure prevailing there acts only on the radially outer annular piston area section. On the opposite side of the second valve body 76, in contrast, the gas pressure acts on the whole piston area. Moreover, through the axial end-side spigots which ensure the axial offset of the piston area sections it is ensured that the end-side cavity regions 42c and 42d are always capable of flow-through regardless of the position of the second valve body 76 as part of the first or the second gas supply line 60 or 64 respectively.

In the depiction of FIG. 8, let it be assumed that to the first source-linking aperture 26 and to the second source-linking aperture 54 there is connected in each case a full cylinder with respiratory gas or oxygen as the case may be for supplying a patient. The gas cylinder connected to the second source-linking aperture 54 was most recently changed. During this replacement process, gas from the cylinder at the first source-linking aperture 26 was supplied via the first gas supply line 60 to the output-linking aperture 18. This supply state is still continuing.

In the first gas supply line 60 and in the second gas supply line 64 there prevail therefore approximately equal gas pressures. Since, however, the gas pressure in the first gas supply line 60 acts in each case on the whole left piston area of the first and the second valve body 74 and 76 respectively, whereas the gas pressure in the second gas supply line 64 acts only on the radially outer annular right piston area section of the first and the second valve body 74 and 76 respectively, there results at each of the two valve bodies 74 and 76 a force which loads and/or tensions respectively each of the valve bodies 74 and 76 to the right into the first operational position shown in FIG. 8 and already taken up.

Preferably, gas cylinders, i.e. individual gas cylinders, are connected to the source-linking apertures 26 and 54 via a pressure reducer 90a, 90b, 90c (s. FIG. 12) which reduces the higher pressures prevailing in the gas cylinders to for example a pressure of 6 bar (600 kPa). This reduced pressure prevailing at the source-linking apertures 26 and 54 remains approximately constant until shortly before complete emptying, before it then begins to drop rapidly on approaching the complete emptying of the cylinder.

The gas-supplying source at the first source-linking aperture 26 in FIG. 8 now dwindles, with the consequence that the gas pressure prevailing in the second gas supply line 64 increases relative to the gas pressure prevailing in the first gas supply line 60. The piston areas of the valve bodies 74 and 76 are so dimensioned that a smaller pressure difference between the first and the second gas supply line 60 and 64 respectively suffices to displace the second valve body 76 between its operational positions than is needed for displacing the first valve body 74 between its operational positions.

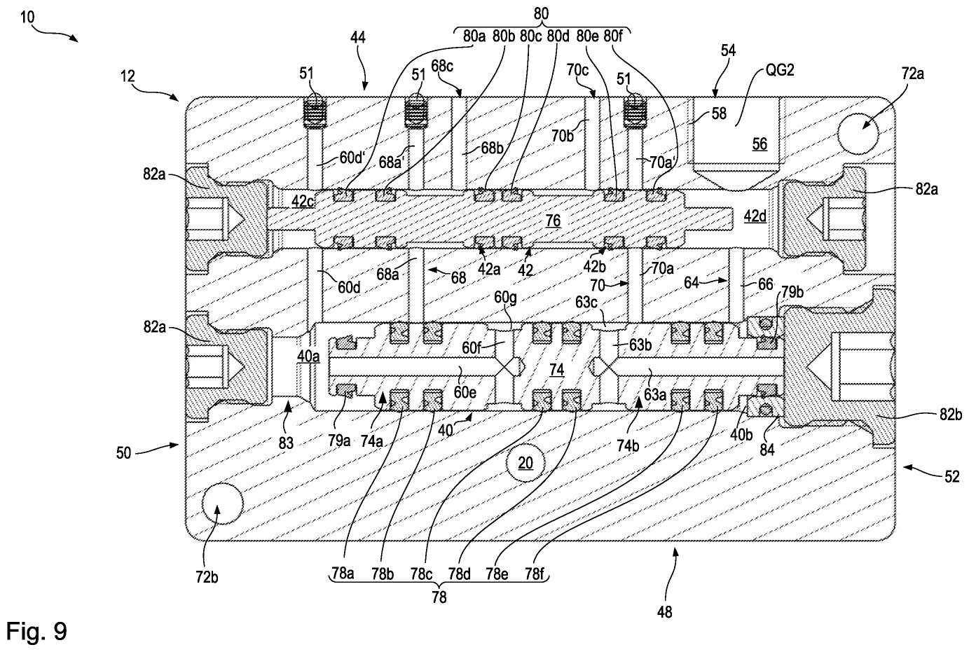

FIG. 9 shows a transitional phase with decreasing gas pressure in the first gas supply line 60, in which the relative overpressure in the second gas supply line 64 with respect to the first gas supply line 60 suffices to displace the second valve body 76 from its first operational position into the second, but does not suffice to also displace the first valve body 74 from its first operational position into the second.

In the second operational position of the second valve body 76 shown in FIG. 9, it blocks the second venting line 70 and opens the first venting line 78, such that when the first valve body 74 is also displaced into the second operational position, gas from the first cavity region 40a can escape via the first venting line 68 to the first sink outlet aperture 68c and thus towards the outside environment. This, however, can only take place once the first valve body 76 has reached its second operational position and thereby connects the first cavity region 40a via the line sections 60e, 60f, and the ring line 60g with the line section 68a.

As soon as the seal 79a seals against the constriction 83, only the radially outer annular piston area section of the left piston area of the first valve body 74 is still acted upon by the in any case decreasing pressure of the first gas supply line 60. The gas then enclosed under continued movement of the first valve body 74 to the left in the region of the line sections 60e, 60f, and 60g configured in it is moved together with the first valve body 74. Only gas enclosed between the radially inner piston area section which is sealed against the constriction 83 and the opposite locking nut 82a is compressed by the movement of the first valve body 74 to the left and ensures an increase in the gas pressure in the line sections 60e, 60f, and 60g inside the first valve body 74.

As soon as the first valve body 74 has reached its second operational position shown in FIG. 10, surplus gas can escape from the line sections 60e, 60f, and 60g inside the first valve body 74 via the first venting line 68 already opened by the second valve body 76.

Since in FIG. 10 both valve bodies 74 and 76 are in the second operational position, FIG. 10 shows the switching-valve assembly 10 in its second operational state. The first gas supply line 60 is now interrupted and the emptied source connected to the first source-linking aperture 26 can be replaced with a fresh, full source. Due to the described division of the piston areas and the in the second operational state on the left side of FIG. 10 by gas in the first gas supply line 60 only reachable radially outer piston area sections, the pressure increase in the first gas supply line 60 effected by the source replacement changes nothing in the operational state of the switching-valve assembly 10. The latter supplies without interruption gas from one of the sources until its emptying and then allows its replacement by a full source.

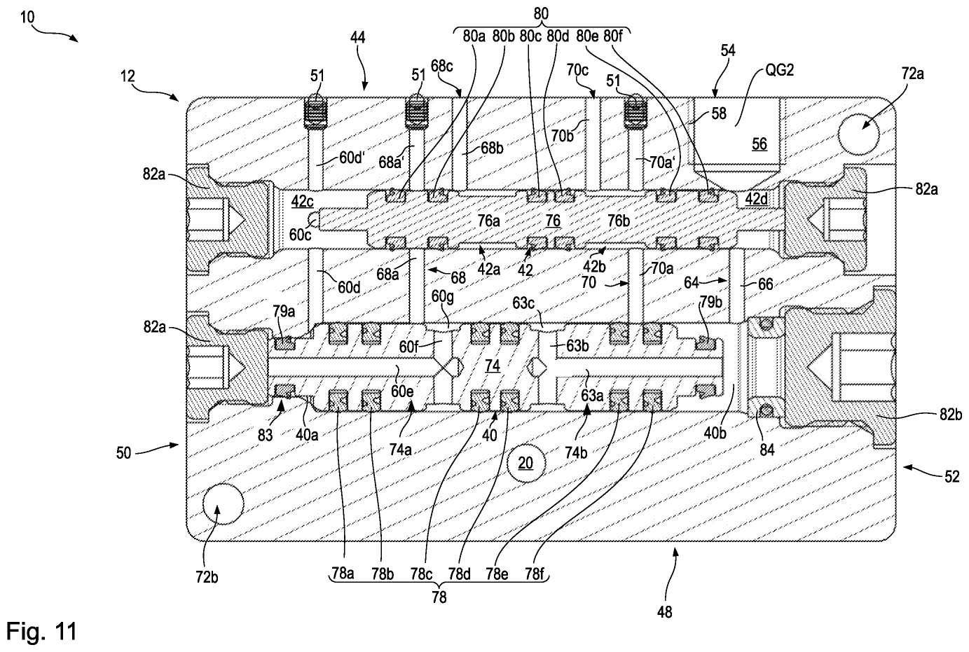

FIG. 11 shows a further transitional phase of the switching-valve assembly 10 which corresponds to the transitional phase of FIG. 9, but this time during a transition from the second operational state into the first operational state shown in FIG. 8.

Under continues emptying of the source connected to the second source-linking aperture 54, the relative increase in the gas pressure in the first gas supply line 60 supplied by a fresh source first ensures an adjustment of the second valve body 76 into the first operational position, in which, as in FIG. 8, the first venting line 68 is interrupted and the second venting line is released for venting of the second cavity region 40b.

FIG. 12 depicts in rough schematic form an example of a facility 2 for supplying a patient P with oxygen. The facility 2 comprises a stack 4 or a bundle 4 with two switching-valve assemblies 10 and a likewise stackable prioritization valve assembly 11, which are stacked along a stack axis SA.

The stack 4 is secured against falling apart by two connecting bolts 86. The connecting bolts 86 are inserted in the passage apertures 72a and 72b of the housing 12 of the switching-valve assemblies 10 and screwed into apertures 73a and 73b in the housing of the prioritization valve assembly 11 which are provided with internal threads (s. FIG. 13).

The lateral surfaces 46 of the housing 12 of the switching-valve assemblies 10 and the lateral surface 47 of the housing 13 of the prioritization valve assembly 11 form a common exterior surface of the stack 4. The housing 13 of the prioritization valve assembly 11 consequently exhibits essentially the same dimensions as the housing 12. It is harmless if the height dimension of the housing 13 along the stack axis SA differs from the height dimension of the housing 12.

To the first source-linking aperture 26 of the top switching-valve assembly 10 in stack 4 there is connected an oxygen cylinder 88a via a line 92a with an intermediate arrangement of a pressure reducer 90a. The pressure reducer 90a reduces the pressure of the gas emerging from the oxygen cylinder 88a to a uniform pressure of for example 6 bar (600 kPa).

To the second source-linking aperture 54 of the top switching-valve assembly 10 in stack 4 there is connected by means of a second line 92b a second oxygen cylinder 88b via a second pressure reducer 90b.

The output-linking aperture 18 of the top switching-valve assembly 10 which is not visible in FIG. 12, is connected directly with the first source-linking aperture 26 as the follow-up linking aperture 26 of the second switching-valve assembly 10 of stack 4, such that the upper switching-valve assembly 10 of stack 4 in FIG. 12 forms the gas source for the first source-linking aperture 26 of the lower switching-valve assembly 10 in stack 4. To the second source-linking aperture 54 of the lower switching-valve assembly 10 in stack 4 there also is connected an oxygen cylinder, namely the oxygen cylinder 88c, which with an intermediate arrangement of a pressure reducer 90c is connected to the second source-linking aperture 54 via the line 92c. Due to the intermediately arranged pressure reducers 90a, 90b, and 90c, the lines 92a, 92b, and 92c always supply a constant pressure of for example 6 bar to the respective connecting apertures, until the respective assigned oxygen cylinders are nearly completely emptied.

The output-linking aperture of the lower switching-valve assembly 10 in FIG. 12 is coupled in stack 4 with a non-depicted first source-linking aperture 27 of the prioritization valve assembly 11, such that the switching-valve assemblies 10 form the first gas source of the prioritization valve assembly 11. To a second source-linking aperture 55 of the prioritization valve assembly 11 there is connected a house line 94 of a clinic K as a gas source. The functionally known per se prioritization valve assembly is so designed that whenever a house line 94 of a clinic K is connected as a gas source to the prioritization valve assembly 11, the gas of the house line 94 is routed to the output-linking aperture of the prioritization valve assembly 11, whereas a gas source connected to the first source-linking aperture 27 of the prioritization valve assembly 11 is only released for delivery at the output-linking aperture 19 of the prioritization valve assembly 11 if the house line 94 is separated from the prioritization valve assembly 11 or, against expectation, should be depleted.

To the output-linking aperture of the prioritization valve assembly 11 there is connected a respiratory gas line 96, which conveys gas supplied to the stack 4 to a respiratory device B, which in turn dispenses the gas to the patient P via an interface 98. In the example of FIG. 12, the interface 98 is depicted as a mask. The interface 98 can, however, also be a nasal cannula.

Through the stack 4, uninterrupted supply to the patient P of desired respiratory gas can be ensured, where an installed respiratory gas source in a clinic has precedence before gas cylinders as respiratory gas sources.

FIG. 13 depicts an exploded view of the stack 4. It shows the first source-linking aperture 27 of the prioritization valve assembly 11, likewise the prioritization valve piston 77 which is displaceable between two operational positions and the locking nut which closes off its valve piston cavity.

The apertures 73a and 73b which align with the passage apertures 72a and 72b respectively in stack engagement exhibit internal threads into which the bolts 86 can be screwed.

FIG. 14 shows a longitudinal section through the stack 4 along a section plane containing the coaxial gas supply paths QG1 and AG and parallel to the longitudinal portions 44 and 48.

FIG. 14 depicts how the gas supply line sections 20 and 28 of the switching-valve assemblies 10 which are stacked with one another, align with one another and form a common continuous gas supply line region 30.

The gas supply line section 28 of the lower switching-valve assembly 10 in FIG. 14, also aligns with the gas supply line section 29 which starting from the first source-linking aperture 27 of the prioritization valve assembly 11 proceeds into its housing 13, and forms with it a common continuous gas supply line region 99.

While considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-18. (canceled)

19. A switching-valve assembly for switching between two respiratory gas sources of a respiratory device, where the switching-valve assembly comprises:
A housing with a first source-linking aperture, with a second source-linking aperture arranged spatially at a distance from the first one and with an output-linking aperture arranged spatially at a distance from the first and from the second source-linking aperture,
A first gas supply line which connects the first source-linking aperture with the output-linking aperture,
A second gas supply line which connects the second source-linking aperture with the output-linking aperture,
A valve arrangement accommodated in the housing which is displaceable between a first operational state in which it releases the first gas supply line for flow-through of respiratory gas and blocks the second gas supply line and a second operational state in which it blocks the first gas supply line and releases the second gas supply line for flow-through of respiratory gas,
where the switching-valve assembly is configured stackably as a modular switching-valve assembly, wherein the housing exhibits a first outside section with a first stack exterior surface section and a second outside section arranged at a distance and/or at an angular distance relative to the first outside section with a second stack exterior surface section configured complementarily to the first stack exterior surface section, where the output-linking aperture is configured at an outside section out of the first and the second outside section and where a source-linking aperture out of the first and the second source-linking aperture is configured as a follow-up linking aperture at the respective other outside section out of the first and the second outside section, namely in such a manner that when the first and the second stack exterior surface sections are conceived as stack exterior surface sections of similar separate modular switching-valve assemblies in stack engagement with one another, gas supply line sections each extending from the outlet- and the follow-up linking apertures into the housing are connected into a continuous gas supply line region.

20. The modular switching-valve assembly according to claim 19, wherein at each outside section out of the first and the second outside section at least one connecting formation is provided, which is configured for securing a physical connection of the modular switching-valve assembly with a similar separate modular switching-valve assembly, where a relative spatial arrangement between the follow-up linking arrangement and the connecting formation in the outside section exhibiting the follow-up linking arrangement, is the same relative spatial arrangement which exists between the output-linking aperture and the connecting formation in the outside section exhibiting the output-linking aperture.

21. The modular switching-valve assembly according to claim 19, wherein the respective other source-linking aperture out of the first and the second source-linking aperture which is not the follow-up linking aperture, is provided in a different outside section of the housing than the first and the second outside section.

22. The modular switching-valve assembly according to claim 19, wherein the first and the second outside section are configured on opposite housing sides facing away from one another.

23. The modular switching-valve assembly according to claim 21, wherein the first and the second outside section are configured on opposite housing sides facing away from one another, and wherein the respective other source-linking aperture is configured in a third outside section, where the third outside section connects the first and the second outside sections with one another.

24. The modular switching-valve assembly according to claim 19, wherein
i) The first stack exterior surface section exhibits a formation out of a projection and a recess and the second stack exterior surface section exhibits the respective other formation out of a projection and a recess complementarily to the formation of the first stack exterior surface section, or that
ii) The first stack exterior surface section and the second stack exterior surface section are each planar stack exterior surface sections.

25. The modular switching-valve assembly according to claim 19, wherein the first and the second outside section are planar outside sections.

26. The modular switching-valve assembly according to claim 19, wherein the housing exhibits a parallelepiped-like outer shape.

27. The modular switching-valve assembly according to claim 19, wherein the valve arrangement exhibits a first valve body and a second valve body different from the first one, where the first and the second valve body are each arranged between the first and the second source-linking apertures on the one hand and the output-linking aperture on the other, where the first valve body is accommodated displaceably in a first valve body cavity and where the second valve body is accommodated displaceably in a second valve body cavity arranged at a distance from the first valve body cavity, where the first and the second valve body are each displaceable between a first operational position assigned to the first operational state and a second operational position assigned to the second operational state, where the switching-valve assembly exhibits a first sealing arrangement effective between the first valve body and the first valve body cavity which in the first valve body cavity defines a first cavity region and a second cavity region different from the first one, where the switching-valve assembly exhibits a second sealing arrangement effective between the second valve body and the second valve body cavity which in the second valve body cavity defines a third cavity region and a fourth cavity region different from the third one, where a cavity region out of the first and third cavity region as first supply cavity region is part of the first gas supply line but not part of the second gas supply line, and where a cavity region out of the second and fourth cavity region as second supply cavity region is part of the second gas supply line but not part of the first gas supply line,
Where the switching-valve assembly exhibits a first venting line which connects the first supply cavity region with a first sink-linking aperture different from the output-linking aperture,
Where the switching-valve assembly exhibits a second venting line which connects the second supply cavity region with a second sink-linking aperture different from the output-linking aperture,
Where that other cavity region out of the first and third cavity region which is not first supply cavity region is as first venting cavity region part of the first venting line but not of the first gas supply line, and where that other cavity region out of the second and fourth cavity region which is not second supply cavity region is as second venting cavity region part of the second venting line but not of the second gas supply line.

28. The modular switching-valve assembly according to claim 27, wherein the first cavity region is the first supply cavity region, the second cavity region is the second supply cavity region, the third cavity region is the first venting cavity region, and the fourth cavity region is the second venting cavity region.

29. The modular switching-valve assembly according to claim 27, wherein a wall surrounding the first venting cavity region surrounds a first venting valve body region and that a wall surrounding the second venting cavity region surrounds a second venting valve body region, where the first venting valve body region is configured to open or to close the first venting line to a gas flow depending on the operational position of the valve body exhibiting the first venting valve body region, and where the second venting valve body region is configured to open or to close the second venting line to a gas flow depending on the operational position of the valve body exhibiting the second venting valve body region.

30. The modular switching-valve assembly according to claim 27, wherein a wall surrounding the first supply cavity region surrounds a first supply valve body region and that a wall surrounding the second supply cavity region surrounds a second supply valve body region, where the first supply valve body region is configured to block or release the first gas supply line to a gas flow depending on the operational position of the valve body exhibiting the first supply valve body region, and where the second supply valve body region is configured to block or release the second gas supply line to a gas flow depending on the operational position of the valve body exhibiting the second supply valve body region.

31. The modular switching-valve assembly according to claim 29, wherein out of the first and the second valve body only the valve body exhibiting the first venting valve body region is configured to open or to close the first venting line to a gas flow, and/or that out of the first and the second valve body only the valve body exhibiting the second venting valve body region is configured to open or to close the second venting line to a gas flow.

32. The modular switching-valve assembly according to claim 30, wherein out of the first and the second valve body only the valve body exhibiting the first supply valve body region is configured to block or release the first gas supply line to a gas flow, and/or that out of the first and the second valve body only the valve body exhibiting the second supply valve body region is configured to block or release the second gas supply line to a gas flow.

33. The modular switching-valve assembly according to claim 30, wherein in the first supply valve body region there is configured a first gas line which is part of the first gas supply line and/or of the first venting line and/or that in the second supply valve body region there is configured a second gas line which is part of the second gas supply line and/or of the second venting line.

34. The modular switching-valve assembly according to one of the claim 30, wherein the first valve body and the second valve body are displaceable between their first and their second operational position only through the gas pressures prevailing in the switching-valve assembly free from mechanical pre-tensioning devices.

35. A switching-valve arrangement, comprising a first switching-valve assembly according to claim 19 and a second switching-valve assembly according to claim 19, where the first stack exterior surface section of the one switching-valve assembly out of first and second switching-valve assembly with the second stack exterior surface section of the respective other switching-valve assembly out of first and second switching-valve assembly are in stack engagement with one another, forming a gas supply line region continuous across the outlet linking arrangement of the one switching-valve assembly and the follow-up linking arrangement of the other switching-valve assembly.

36. A respiratory device for supplying a patient with respiratory gas, comprising a respiratory gas entry, a respiratory gas exit, a respiratory gas line which connects the respiratory gas entry with the respiratory gas exit, a flow modification device for modifying the respiratory gas quantity flowing through the respiratory gas exit per unit of time and/or for modifying the respiratory gas pressure at the respiratory gas exit, and a control device for controlling the operation of the flow modification device, wherein upstream of the respiratory gas entry the output-linking aperture of at least one switching-valve assembly according to claim 19 is connected with the respiratory gas entry of the respiratory device in a gas-conveying manner.

37. A respiratory device for supplying a patient with respiratory gas, comprising a respiratory gas entry, a respiratory gas exit, a respiratory gas line which connects the respiratory gas entry with the respiratory gas exit, a flow modification device for modifying the respiratory gas quantity flowing through the respiratory gas exit per unit of time and/or for modifying the respiratory gas pressure at the respiratory gas exit, and a control device for controlling the operation of the flow modification device, wherein upstream of the respiratory gas entry the output-linking aperture of at least one switching-valve arrangement according to claim 35 is connected with the respiratory gas entry of the respiratory device in a gas-conveying manner.
